(19) United States
(12) Patent Application Publication
(10) Pub. No.: US 2019/0201252 A1
(43) Pub. Date: Jul. 4, 2019

(54) LIGHT INCONTINENCE PAD

(71) Applicant: ATTENDS HEALTHCARE PRODUCTS, INC., Greenville, NC (US)

(72) Inventors: Alice Tilson KOEHLER, Greenville, NC (US); Charles F. SCHROER, Jr., Greenville, NC (US); John Patrick COSTELLO, Oxford, OH (US)

(73) Assignee: ATTENDS HEALTHCARE PRODUCTS, INC., Greenville, NC (US)

(21) Appl. No.: 16/322,805

(22) PCT Filed: Jun. 8, 2018
(86) PCT No.: PCT/US2018/036641

§ 371 Date: Feb. 1, 2019

Related U.S. Application Data
(60) Provisional application No. 62/517,351, filed on Jun. 9, 2017.

(51) Int. Cl. A61F 13/476; A61F 13/472; A61F 13/475; A61F 13/511; A61F 13/514; A61F 13/56

(52) U.S. Cl. CPC A61F 13/476; A61F 13/472; A61F 13/475; A61F 2013/15121; A61F 13/514; A61F 13/5611; A61F 13/511

(57) ABSTRACT

Disposable absorbent articles, such as adult pads or liners for light incontinence, with reduced lengths for improved fit and comfort.

### CROSS REFERENCE TO RELATED APPLICATIONS

This application claims the benefit of priority to U.S. Provisional Patent Application No. 62/517,351 filed Jun. 9, 2017, the contents of which application are incorporated by reference in their entirety.

### FIELD OF INVENTION

The present invention relates generally to incontinence pads and sanitary napkins; and more particularly, but not by way of limitation, to female incontinence pads or liners that are smaller and thinner to provide protection specifically for light incontinence with improved fit, comfort, and discretion.

### BACKGROUND

Examples of disposable absorbent articles that are wearable by a user include baby diapers, training pants, adult incontinence briefs and underwear, bladder control pads, and feminine hygiene articles, all of which may be made in disposable forms. “Disposable” refers to articles that are designed to be discarded after a limited use rather than being laundered or otherwise restored for reuse. Disposable absorbent products have met with widespread acceptance in the marketplace for a variety of applications, including infant and adult incontinence care, in view of the manner in which such products can provide effective and convenient liquid absorption and retention while maintaining the comfort of the wearer. Such disposable absorbent articles often include a topsheet that is configured to be closest to the wearer during use, a liquid-impermeable backsheet or outer cover, and an absorbent core between the topsheet and the backsheet. In some instances, such disposable absorbent articles also include an acquisition-distribution layer (ADL) disposed between the topsheet and the absorbent core.

Such disposable absorbent articles also include, for example, pads and liners for feminine hygiene and light incontinence uses, i.e., pads and liners adapted to absorb urine and/or blood. Many women suffer from light urinary incontinence. Most protective, absorbent pads available on the market today for female bladder control issues or menstruation management are designed in an oval shape or a dog-bone shape, primarily to afford coverage over both urethral area and vaginal area. One example of such a pad or liner is disclosed in U.S. Patent Application Publication No. US 2001/0014797.

Some such liners are shaped for certain types of undergarments, such as thong or t-back underwear. Examples of such liners are disclosed, for example, in U.S. Patent Application Publication No. 2004/0102747, and U.S. Patent Application Publication No. US 2002/0115978.

Some such liners are folded to form a desired shape. One example of such a liner is disclosed in U.S. Pat. No. 6,443,934.

### SUMMARY

This disclosure includes embodiments of disposable absorbent articles, particularly pads or liners adapted for light-incontinence applications. Embodiments of the present pads are shaped to improve fit, comfort and discretion, such as, for example, by reducing size and potential for bunching. By way of example, embodiments of the present pads and lines are configured to be co-located with, for example extend under, a wearer's urethra, but not over the entirety of the wearer's vagina, thereby different in size and shape relative to prior art pads and liners, which are typically adapted to fit far enough posteriorly to extend under the entirety of the wearer's vagina. While the prior art approach may be considered necessary to provide sufficient protection and absorbency for feminine hygiene applications, such size is unnecessary for user's needing only light incontinence protection. The present pads and liners can therefore be configured to provide such light incontinence protection, while reducing the overall size of the pad or liner to improve comfort and discretion during use. In some embodiments, the present pads have a limited absorbent capacity, such as, for example, an absorbent capacity in grams of water or of saline of 150 g or less, 120 g or less, 100 g or less, 80 g or less, or 60 g or less.

Some embodiments of present disposable absorbent articles comprise: a pad configured to be inserted into a crotch portion of a wearer's garment or undergarment, where the pad comprises: a liquid-permeable topsheet; a liquid-impermeable backsheet; and an absorbent core disposed between the topsheet and the backsheet.

In some embodiments of the present pads, the pad has a body-facing surface defined by the topsheet, an outer surface defined by the backsheet, an anterior end, a posterior end, a perimeter, a length measured along a central longitudinal axis of the pad between the anterior and posterior ends, and a maximum width measured perpendicular to the length along the body-facing surface, the maximum width disposed closer to the anterior end than to the posterior end; the anterior end includes a first lobe on a left side of the central longitudinal axis, and a second lobe on a right side of the central longitudinal axis; and the posterior end extends from the anterior end by a distance that is greater than the maximum width. In some embodiments, a portion of the perimeter defining the anterior end includes: a first arc on a left side of the pad, and a second arc on a right side of the pad, where the first arc and second arc are separated along the perimeter on the anterior end by at least one additional arc or line. In some embodiments, a portion of the perimeter defining the posterior end includes a third arc that spans the central longitudinal axis. In some embodiments, the perimeter includes a left side segment extending between the first arc and the posterior end, and a right side segment extending between the second arc and the posterior end. In some embodiments, the left and right side segments are concave toward the central longitudinal axis. In some embodiments, the posterior end defines a third lobe.

In some embodiments of the present pads, the pad has a body-facing surface defined by the topsheet, an outer surface defined by the backsheet, an anterior end, a posterior end, a perimeter, a length measured along a central longitudinal axis of the pad between the anterior and posterior ends, and a maximum width measured perpendicular to the length, the maximum width disposed closer to the anterior end than to the posterior end; and the posterior end extends from the anterior end to define the length; and the length is 1 to 2.5 times the maximum width. In some embodiments, the length is 1.75 to 2.25 times the maximum width, and the maximum width is closer to the anterior end than to the posterior end. In some embodiments, the length is 1 to 1.5 times the maximum width. In some embodiments, the maximum width is disposed at a distance from the anterior end of 1%-25% of the length of the pad. In some embodiments, the maximum width is disposed at a distance from the anterior end of 15%-20% of the length of the pad. In some embodiments, the maximum width is disposed at a distance from the anterior end of 5%-10% of the length of the pad.

In some embodiments of the present pads, the absorbent core comprises a laminate of superabsorbent polymer (SAP) particles sandwiched between a first layer and a second layer, where at least one of the first and second layers is hydrophilic. In some embodiments, the laminate is folded.

Some embodiments of the present pads further comprises: an acquisition-distribution layer (ADL) disposed between the topsheet and the absorbent core.

In some embodiments of the present pads, the pad has a body-facing surface defined by the topsheet, an outer surface defined by the backsheet, an anterior end, a posterior end, a perimeter, a length measured along a central longitudinal axis of the pad between the anterior and posterior ends, and a maximum width measured perpendicular to the length, the maximum width disposed closer to the anterior end than to the posterior end; and a first portion of the backsheet on a left side of the pad is folded over the posterior end, and a second portion of the backsheet on a right side of the pad is folded over the posterior end, such that the first and second portions of the backsheet cooperate with the topsheet to define a pocket extending from the posterior end and opening toward the anterior end of the pad. In some embodiments, the first portion of the backsheet is bonded to the second portion of the backsheet. In some embodiments, the absorbent core comprises a laminate of superabsorbent polymer (SAP) particles sandwiched between a first layer and a second layer, where at least one of the first and second layers is hydrophilic. In some embodiments, the laminate is folded. Some embodiments further comprise: an acquisition-distribution layer (ADL) disposed between the topsheet and the absorbent core.

In some embodiments of the present pads, the absorbent core has a maximum thickness and defines one or more channels each having a thickness that is less than the maximum thickness. In some embodiments, the maximum thickness is defined by a plurality of layers of an absorbent sheet or web. In some embodiments, the one or more channels are each defined by embossed lines or regions at which the plurality of layers are compressed.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the term “substantially” may be substituted with “within [a percentage] of” what is specified, where the percentage includes 0.1, 1, 5, and 10 percent.

The terms “comprise” and any form thereof such as “comprises” and “comprising,” “have” and any form thereof such as “has” and “having,” and “include” and any form thereof such as “includes” and “including” are open-ended linking verbs. As a result, an apparatus that “comprises,” “has,” or “includes” one or more elements possesses those one or more elements, but is not limited to possessing only those elements. Likewise, a method that “comprises,” “has,” or “includes” one or more steps possesses those one or more steps, but is not limited to possessing only those one or more steps.

Any embodiment of any of the apparatuses, systems, and methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Further, a device or system that is configured in a certain way is configured in at least that way, but it can also be configured in other ways than those specifically described.

The feature or features of one embodiment may be applied to other embodiments, even though not described or illustrated, unless expressly prohibited by this disclosure or the nature of the embodiments.

Some details associated with the embodiments described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. Views in the figures are drawn to scale, unless otherwise noted, meaning the sizes of the depicted elements are accurate relative to each other for at least the embodiment in the view.

FIG. 1A depicts a top plan view of an embodiment of the present incontinence pads.

FIG. 1B depicts a bottom plan view the pad of FIG. 1A.

FIG. 2 depicts an exploded, lateral cross-sectional view of the pad along line 2-2 of FIG. 1A.

FIG. 3A depicts a top plan view of a second embodiment of the present incontinence pads in which the absorbent core includes a channel.

FIG. 3B depicts a cross-sectional view of a first alternate absorbent core that is suitable for use in the pad of FIG. 3A.

FIG. 3C depicts a cross-sectional view of a second alternate absorbent core that is suitable for use in the pad of FIG. 3A.

FIG. 3D depicts a cross-sectional view of a third alternate absorbent core that is suitable for use in the pad of FIG. 3A.

FIG. 3E depicts a cross-sectional view of a fourth alternate absorbent core that is suitable for use in the pad of FIG. 3A.

FIG. 3F depicts a cross-sectional view of a fifth alternate absorbent core that is suitable for use in the pad of FIG. 3A.

FIG. 4 depicts a perspective view of the pad of FIG. 1A shown in combination with an undergarment worn by a wearer.

FIG. 5 depicts a cross-sectional view of a portion of female anatomy with the pad of FIG. 1A positioned for use and shown in a longitudinal cross-section along line 3-3 of FIG. 1B.

FIG. 6 depicts a top plan view of a second embodiment of the present pads.

FIG. 7 depicts a top plan view of a third embodiment of the present pads.

FIGS. 8A and 8B depict an embodiment of the present methods for forming the pad of FIG. 7.

FIGS. 9A and 9B depict a second embodiment of the present methods form forming a first alternate configuration of the present pads with a posterior pocket.

FIGS. 10A and 10B depict a second embodiment of the present methods form forming a second alternate configuration of the present pads with a posterior pocket.

FIG. 11A depicts an upper plan view of a design of an upper side of the present pads.

FIG. 11B depicts an upper plan view of another design of an upper side of the present pads, in which the sealed edge is shown in dashed lines.

FIG. 11C depicts an upper plan view of another design of an upper side of the present pads, in which the longitudinal boundaries of a central channel are shown in dashed lines.

FIG. 11D depicts an upper plan view of another design of an upper side of the present pads, in which the sealed edge and the longitudinal boundaries of a central channel are shown in dashed lines.

FIG. 11E depicts a lower plan view of a design of a lower side of the present pads, with a multi-part release layer with partially-overlapping portions.

FIG. 11F depicts a lower plan view of another design of a lower side of the present pads, with a boundary between portions of a multi-part release layer shown in dashed lines.

### DETAILED DESCRIPTION OF ILLUSTRATIVE EMBODIMENTS

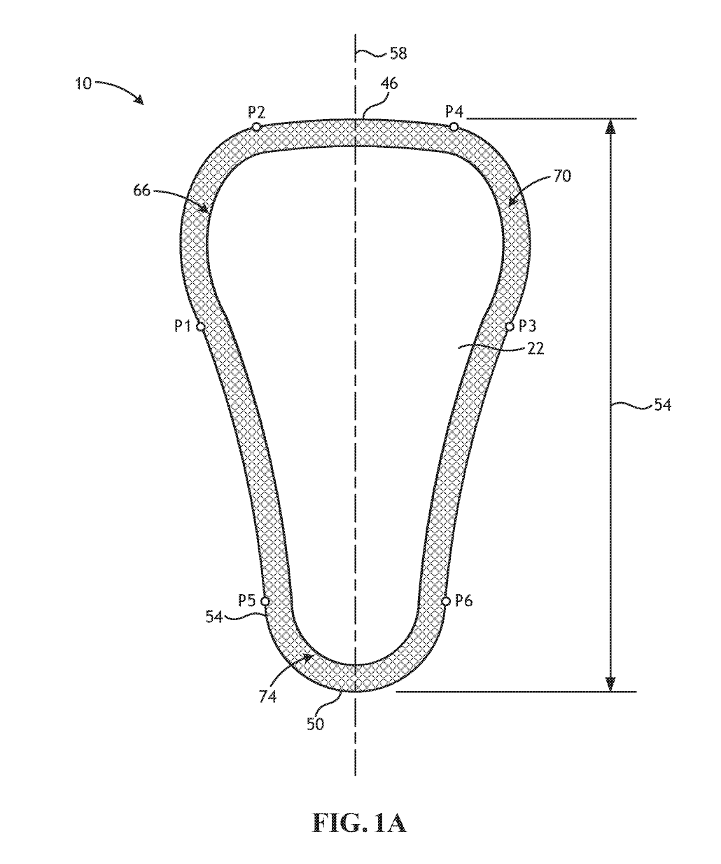

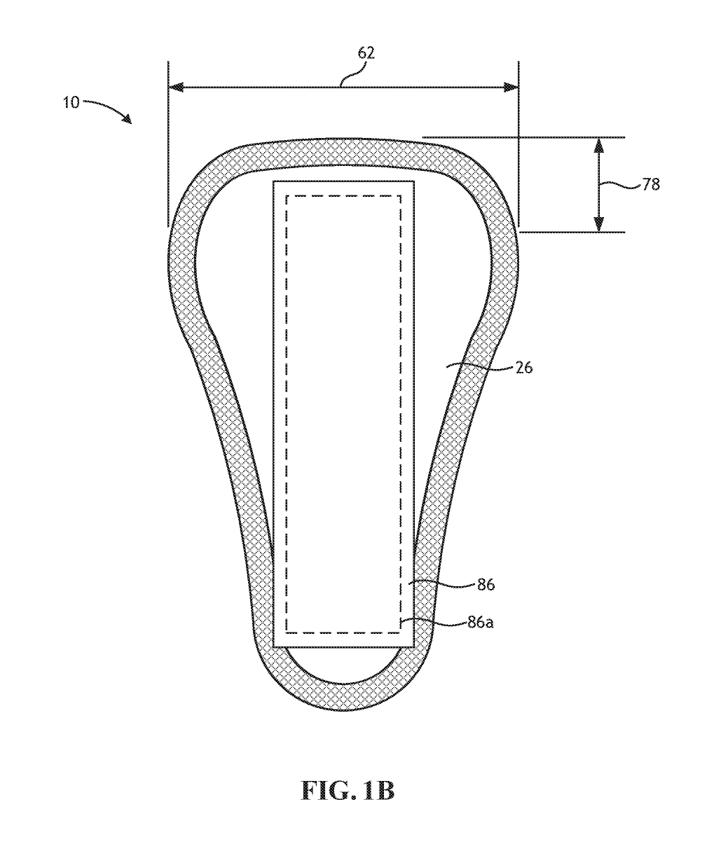

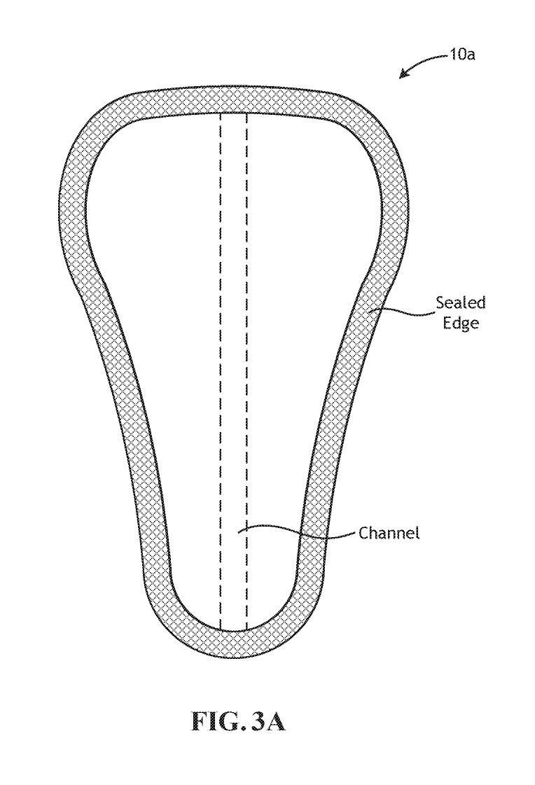

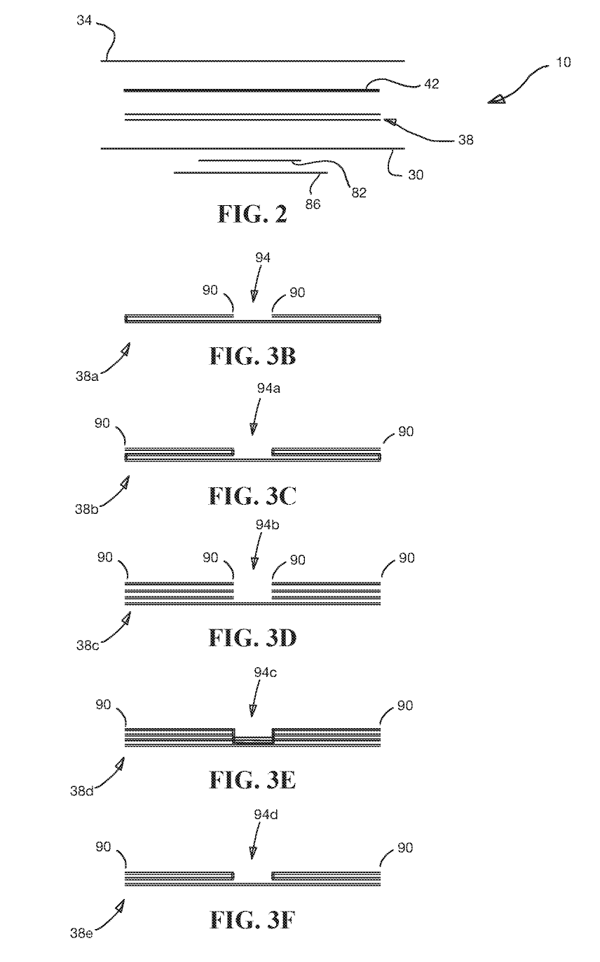

Referring now to the figures and, more particularly, to FIGS. 1A-1B and FIG. 2; FIG. 1A depicts a top plan view of an embodiment 10 of the present incontinence pads; FIG. 1B depicts a bottom plan view of pad 10; and FIG. 2 depicts an exploded, lateral cross-sectional view of pad 10 taken along line 2-2 of FIG. 1A. As shown, pad 10 has a length 14, a maximum width 18, a body-facing surface 22, and an outer surface 26. In this embodiment, body 10 comprises a liquid-impermeable backsheet or cover 30 that defines outer surface 26, a liquid-permeable topsheet 34 that defines body facing surface 22 and is configured to be closest to the wearer during use, an absorbent core 38 disposed between the backsheet and topsheet, and an acquisition-distribution layer (ADL) 42 disposed between the topsheet and the absorbent core. Other embodiments may omit ADL 42. “Liquid impermeable,” when used in describing a layer or multi-layer laminate, means that a liquid, such as urine, will not pass through the layer or laminate, under ordinary use conditions, in a direction generally perpendicular to the plane of the layer or laminate at the point of liquid contact. “Lamination” is the technique of manufacturing a material in multiple layers, so that the composite material has benefits of all the combined layers, such as, for example, improved mechanical strength or durability, improved stability, lower permeability to water, and/or other properties. A laminate includes two or more layers of material(s) that are a permanently assembled by heat, pressure, welding, or adhesives.

As shown in FIGS. 1A and 1B, pad 10—in the depicted flattened or unfolded configuration—has a generally planar configuration. As shown, pad 10 has an anterior end 46, a posterior end 50, and a perimeter 54 along which topsheet 34 is bonded to backsheet 38. As also shown in FIGS. 1A and 1B, length 14 reflects the maximum dimension of the pad measured along a central longitudinal axis 58 of the pad between the anterior and posterior ends, and width 18 is the maximum dimension measured perpendicular to the length along the body-facing surface. In this embodiment, maximum width 18 is disposed closer to anterior end 46 than it is to posterior end 50.

The depicted embodiment of pad 10 has a tri-lobal configuration. More particularly, in the embodiment shown, anterior end 46 includes a first lobe 66 on a left side of the central longitudinal axis, a second lobe 70 on a right side of the central longitudinal axis, and posterior end 50 defines a third lobe 74. By way of example, in this embodiment, a portion of perimeter 54 defining anterior end 46 includes: a first arc, between points P1 and P2, on a left side of the pad; and a second arc, between points P3 and P4, on a right side of the pad that is separated along perimeter 54 by at least one additional arc or line between points P2 and P4. In this embodiment, a portion of perimeter 54 defining posterior end 50 includes a third arc, between points P5 and P6, that spans central longitudinal axis 58.

In the embodiment shown in FIGS. 1A and 1B, perimeter 54 also includes a left side segment that extends between the first arc at point P1 and the posterior end, and a right side segment extending between the second arc at point P3 and the posterior end. For example, as shown, the left side segment includes an arc extending between the first arc at point P1 and the third arc at point P5, and the right side segment includes an arc extending between the second arc at point P3 and the third arc at point P6. In this embodiment, the left side segments includes a fourth arc that is concave toward central longitudinal axis 58, and the right side segment includes a fifth arc that is concave toward central longitudinal axis 58. In some embodiments, the radius of the first arc is equal to the radius of the second arc and/or equal to the radius of the third arc. In some embodiments, the radius of the fourth arc is equal to the radius of the fifth arc, and the radius of the fourth arc is larger than the radius of the first arc.

In the embodiment shown in FIGS. 1A and 1B, core 38 has a perimeter 62 that is proportionally smaller than perimeter 54 of the overall pad such that the shortest distance between perimeter 62 of core 38 and perimeter 54 of pad 10 is substantially constant around perimeter 54. Stated another way, at any given point along perimeter 54, the shortest distance to perimeter 62 is substantially the same. In other embodiments, perimeter 62 of core 38 may have a shape, for example rectangular, that differs from the shape of perimeter 54 of the core.

As also described in more detail below, pad 10 is shaped such that anterior end 46 and, specifically maximum width 18 of pad 10, contours to a wearer's legs to resist rearward movement of the pad relative to the wearer, while posterior end 50 of the pad extends rearward under the wearer's urethra but not under the entirety of the wearer's vagina. For example, in the depicted embodiment of pad 10, posterior end 50 is sized to not extend under any portion of the wearer's vagina. By configuring pad 10 to avoid posterior end 50 from, in use, extending under the entirety of the wearer's vagina, the size of pad 10 is reduced relative to prior art pads, resulting in reduced bunching and increased wearer comfort. This purpose can be achieved, for example, by selecting the ratio of length 14 relative to maximum width 18 and the position of maximum width 18 relative to anterior end 46. For example, in some embodiments, length 14 is 1 to 2.5 times maximum width 18, and/or maximum width 18 can be disposed at a distance from anterior end 46 that is 1% to 25% of length 14. In the embodiment shown in FIGS. 1A and 1B, length 14 is 1.75 to 2.25 times maximum width 18, and maximum width 18 is disposed at a distance 78 from anterior end 46 that is 15% to 20% of length 14. For example, length 14 may be equal to any one of, or between any two of: 75 mm, 100 mm, 125 mm, and/or 150 mm; and maximum width 18 may be equal to any one of, or between any two of: 40 mm, 50 mm, 60 mm, 70 mm, 80 mm, 90 mm, and/or 100 mm.

In the embodiment shown in FIGS. 1A-1B and FIG. 2, pad 10 also includes an adhesive 82 covered by a release strip 86. As will be appreciated by those of ordinary skill in the art, the release strip is configured to protect the adhesive until a wearer is ready to use the pad, at which time the release strip can be peeled away and removed from the adhesive by the user to expose the adhesive. The user can then position the pad in the user's garment or undergarment with the adhesive facing a crotch portion of the garment or undergarment, such that the adhesive will resist slippage of the pad relative to the garment or undergarment. In other embodiments, in addition to or as an alternative to adhesive 82 on the outer side of the pad, the pad can include adhesive on a body-facing side of the pad (the topsheet) to contact a user's skin to resist displacement of the pad relative to the user during use.

Liquid-impermeable backsheet 30 can include, for example, an inner liquid-impermeable film and an outer nonwoven backsheet that can be a nonwoven fabric. A “film” is a membrane-like layer of material formed of one or more polymers, which does not have a form consisting predominately of a web-like structure of fibers and/or other fibers. In some embodiments of the present articles, backsheet or outer cover 30 can be breathable, for example, an inner liquid-impermeable film of backsheet 30 can comprise a breathable film. The terms “breathable,” “breathable film,” “breathable laminate” or “breathable outer cover material” or “breathable backsheet” refers to a film, laminate, or outer cover material having a water vapor transmission rate (“WVTR”) of at least about 300 grams/m²/24 hours. Breathable materials typically rely on molecular diffusion of vapor, and are substantially liquid impermeable. “Nonwoven” fabrics, according to an INDA definition, are broadly defined as sheet or web structures bonded together by entangling fiber or filaments (and by perforating films) mechanically, thermally, or chemically. They are flat, porous sheets that are made directly from separate fibers or from molten plastic or plastic film. They are not made by weaving or knitting and do not require converting the fibers to yarn. The basis weight of nonwoven fabrics is usually expressed as gsm or grams per square meter. “Nonwoven backsheet” is a backing substrate layer in the outer cover; a nonwoven backsheet is most often a nonwoven layer facing away from the wearer.

As shown in FIG. 2, absorbent core 38 is disposed between backsheet 30 and topsheet 34, and ADL 42 is disposed between topsheet 34 and absorbent core 38. An “absorbent core” is a structure typically disposed between a topsheet and backsheet of an absorbent article and containing materials like SAP and/or cellulosic fibers that are configured to absorb liquid in the absorbent article. In some embodiments, the absorbent core (38) may also include a cover layer or envelope material. The absorbent core, and/or the cover layer or envelope material, may comprise: nonwovens, SAP, cellulosic or non-cellulosic materials, films, fibers, or one or more substrates made of any one two or all of these combination materials. “Superabsorbent” or “superabsorbent material” or “SAP” refers to a water-swellable, water-insoluble organic or inorganic material capable, under the most favorable conditions, of absorbing at least about 15 times its weight in an aqueous solution containing 0.9 weight percent sodium chloride and, more desirably, at least about 30 times its weight in an aqueous solution containing 0.9 weight percent sodium chloride and, even more desirably, at least about 50 times its weight in an aqueous solution containing 0.9 weight percent sodium chloride. The SAP materials can be natural, synthetic and modified natural polymers and materials. In addition, the SAP materials can be inorganic materials, such as silica gels, or organic compounds such as cross linked polymers. “Layer” when used in the singular can be a single element or a plurality of elements. For example, a plurality of sheets may together define a single layer, such as, for example, a layer with a particular function to which the sheets of the layer contribute.

Specifically, in the embodiment shown, absorbent core 38 comprises a laminate of superabsorbent polymer (SAP) particles sandwiched between a first layer and a second layer, where at least one of the first and second layers is hydrophilic; in some embodiments, both layers of the laminate are hydrophilic. In this embodiment, the absorbent core does not include fluff pulp and may therefore be referred to as a “pulp-less core”). Other embodiments can include an absorbent core with pulp alone or pulp and SAP.

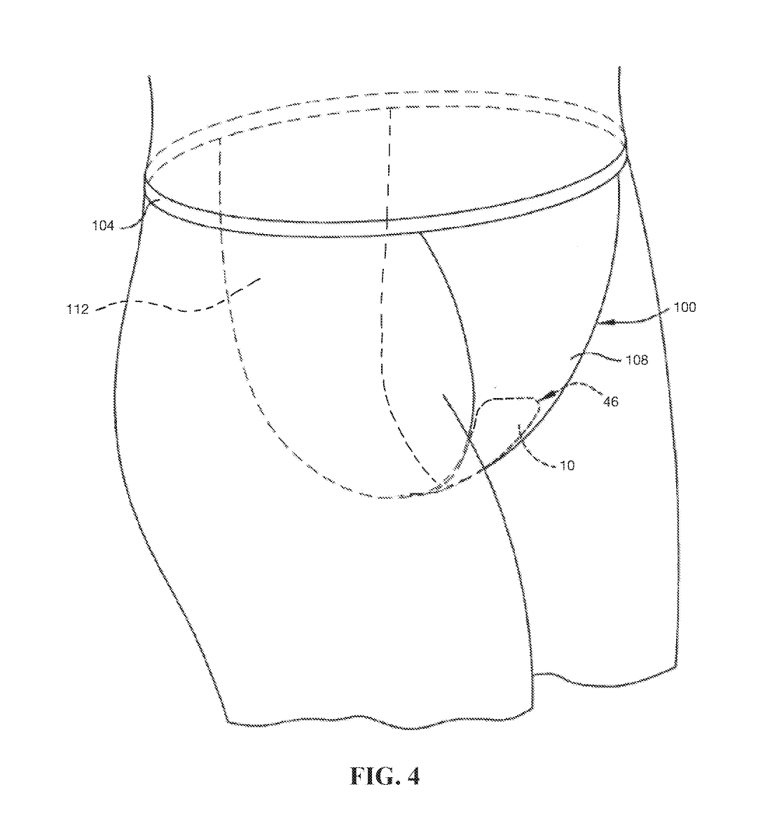

Referring now to FIGS. 3A-3E, FIG. 3A depicts a top plan view of a second embodiment 10a of the present incontinence pads, and FIGS. 3B-3E depict cross-sectionals taken along the line 3-3 of FIG. 3A of various alternate embodiments of absorbent cores suitable for use in pad 10a. Pad 10a is substantially similar to pad 10, with the exception that certain aspects of the absorbent core of pad 10a, specifically that the absorbent core includes one or more channels 94 as described in more detail below.

FIG. 3B depicts a cross-sectional view of first alternate embodiment of an absorbent core 38a that is suitable for use in at least some embodiments of the present pads. Absorbent core 38a is substantially similar to absorbent core 38, with the exception that absorbent core 38a is C-folded. Specifically, as shown, end portions 90 of the laminate are folded over toward a centerline of the laminate to define a central channel 94 that can receive and at least partially trap fluids insulting the core. FIG. 3C depicts an additional embodiment 38b of a folded absorbent core in which end portions 90 of the laminate are folded twice—once towards the centerline, and again back away from the centerline to define three layers of laminate on each side of channel 94a. Other embodiments can be folded any number of times. Specific additional examples of folded cores that may be suitable for certain embodiments of the present pads are disclosed in U.S. Patent Application Publication No. US 2015/0245958, which is incorporated by reference in its entirety.

FIGS. 3D and 3E depict additional examples 38c, 38d of the present absorbent cores that include multiple pieces of the absorbent laminate rather than a single piece folded to define multiple layers. Specifically, core 38c of FIG. 3D includes a lowermost piece of laminate spanning the entire area of the core, and a plurality of smaller pieces of laminate to increase the thickness of the core on either side of channel 94b. This configuration results in end portions of each layer both at the lateral edges of the core and, for the layers above the lower-most layer, bounding channel 94b. As shown, core 38c includes a lowermost layer spanning the entire area of the core, and three partial layers above the lowermost layer; but, in other configurations, the core can include, for example, one such partial layer, two such partial layers, four such partial layers, five such partial layers, or more. The pieces of laminate may, for example, be bonded together by adhesive and/or via embossing. For example, the core in FIG. 3E includes a plurality of sheets of laminate spanning the full width of core 38d, such that ends 90 are disposed only at the lateral edges of the core, and a line of embossing defines channel 94c along a line or series of points at which the sheets are compressed. In the embodiment of FIG. 3E, the sheets may be joined by the embossing alone, or may be bonded with adhesive as well. In some embodiments, the pieces of laminate are die cut before being assembled; in other embodiments, the pieces of laminate are assembled, for example by folding a single piece of laminate, and then the overall core is die cut from the assembled layers.

FIG. 3F depicts a further example of a core 38e that is substantially similar to core 38b of FIG. 3C with the exception that, after being folded the second time, core 38e is die cut or otherwise trimmed to remove the outer pair of folds.

While each of FIGS. 3A-3F depict a single longitudinal channel 94, other embodiments of the present cores and pads including such cores may include multiple longitudinal channels, a single lateral channel, or multiple lateral channels.

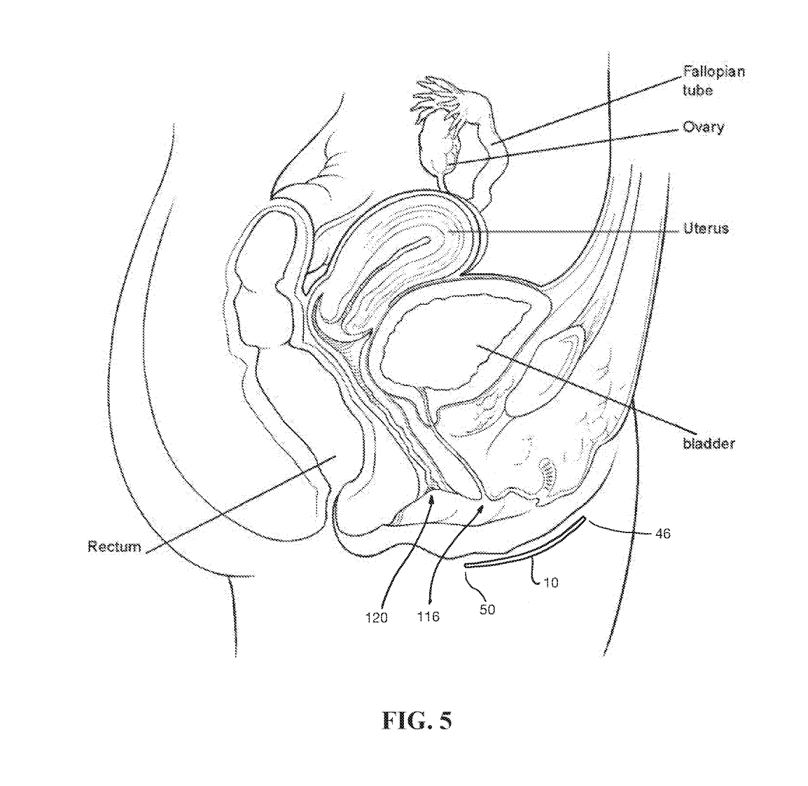

Referring now to FIGS. 4 and 5, FIG. 4 depicts a perspective view of pad 10 shown in combination with an undergarment 100 worn by a wearer, and FIG. 5 depicts a cross-sectional view of a portion of female anatomy with pad 10 positioned for use and shown in a longitudinal cross-section. As shown, undergarment 100 includes a waist band 104, a front or crotch portion 108, and a rear portion 112. As also shown, pad 10 is positioned in crotch portion 108 with anterior end 46 facing forward. As shown in FIG. 5, pad 10 is shaped to permit posterior end 50 to extend to a point that is behind a wearer's urethra 116 but not all the way behind the user's vagina 120. For example, posterior end 50 may not extend under the user's vagina at all, or may extend only part of the way under the user's vagina.

As also described in more detail below, pad 10 is shaped such that anterior end 46 and, specifically maximum width 18 of pad 10, contours to a wearer's legs to resist rearward movement of the pad relative to the wearer, while posterior end 50 of the pad extends rearward under the wearer's urethra but not under the entirety of the wearer's vagina. For example, in the depicted embodiment of pad 10, posterior end 50 is sized to not extend under any portion of the wearer's vagina. By configuring pad 10 to avoid posterior end 50 from, in use, extending under the entirety of the wearer's vagina, the size of pad 10 is reduced relative to prior art pads, resulting in reduced bunching and increased wearer comfort. This purpose can be achieved, for example, by selecting the ratio of length 14 relative to maximum width 18 and the position of maximum width 18 relative to anterior end 46. For example, in some embodiments, length 14 is 1 to 2.5 times maximum width 18, and/or maximum width 18 can be disposed at a distance from anterior end 46 that is 1% to 25% of length 14. In the embodiment shown in FIGS. 1A and 1B, length 14 is 1.75 to 2.25 times maximum width 18, and maximum width 18 is disposed at a distance 78 from anterior end 46 that is 15% to 20% of length 14.

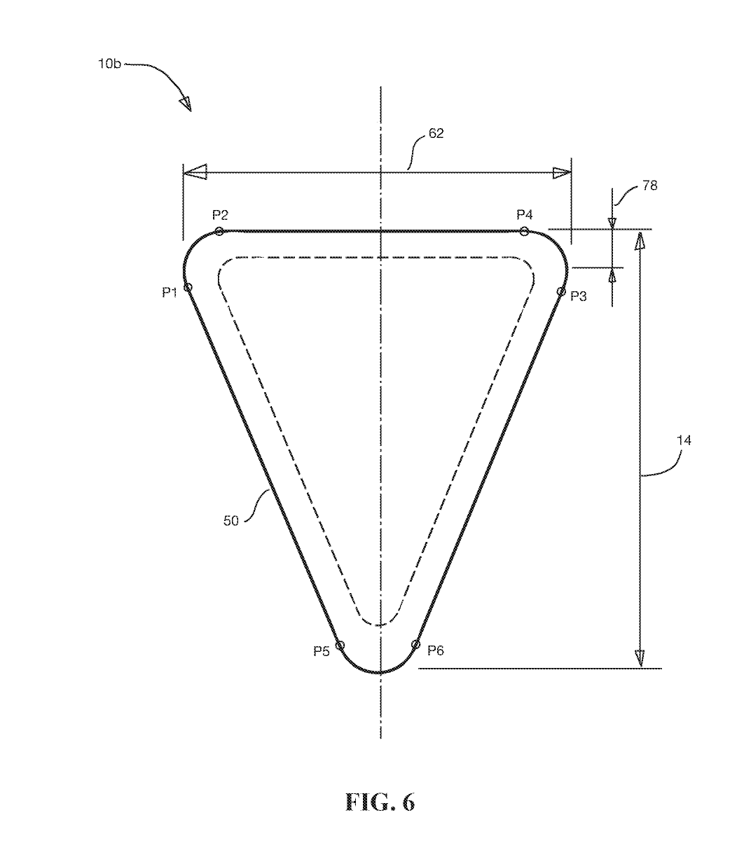

FIG. 6 depicts a top plan view of a second embodiment 10b of the present pads. Pad 10b is substantially similar to pad 10, with the exception that certain aspects of the shape of pad 10b differ relative to pad 10. For example, pad 10b has a different ratio of length 14 to maximum width 18, maximum width 18 is disposed closer to anterior end 46, and the left and right side segments of pad 10b include straight lines instead of arcs. Specifically, length 14 of pad 10b is 1 to 1.5 times maximum width 18, and maximum width 18 is disposed at a distance 78 from anterior end 46 of 5%-10% of length 14 of the pad. Additionally, in the embodiment shown in FIG. 6, perimeter 50 pad 10b includes a left side segment that is a straight line extending between points P1 and P5, and a right side segment that is a straight line extending between points P3 and P6.

FIG. 7 depicts a top plan view of a third embodiment 10c of the present pads. Pad 10c is substantially similar to pad 10b, with the exception that pad 10c includes a pocket 200 defined at or over at least a portion of posterior end 50 to assist with temporarily retaining liquid while the liquid is absorbed by the absorbent core. Specifically, in the embodiment shown, a first portion 204 of backsheet 30 on a left side of the pad is folded over posterior end 50, and a second portion 208 of backsheet 30 on a right side of the pad is folded over posterior end 50, such that the first and second portions 204, 208 of the backsheet cooperate with topsheet 34 to define pocket 200 extending from posterior end 50 and opening toward anterior end 46 of the pad. First portion 204 can, for example, be bonded to second portion 208, such as via adhesive, one or more ultrasonic bonds, and/or the like, to retain the pocket shape.

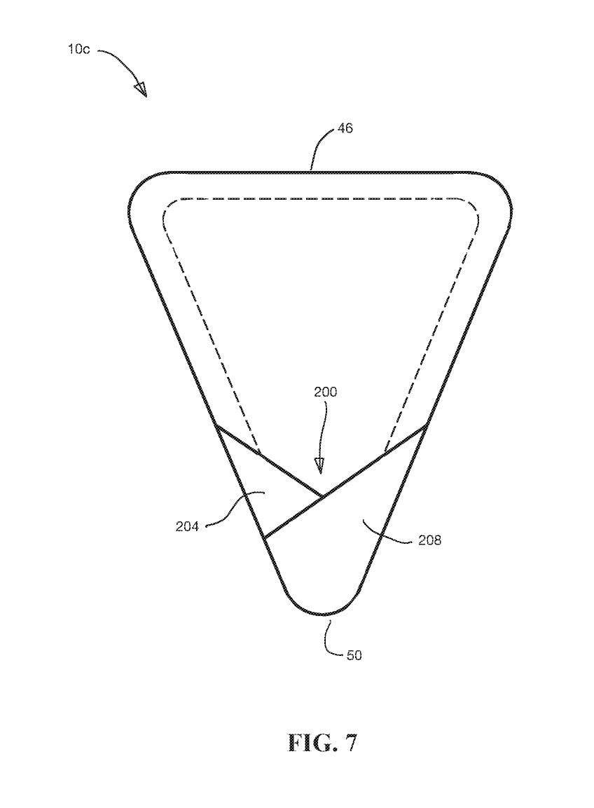

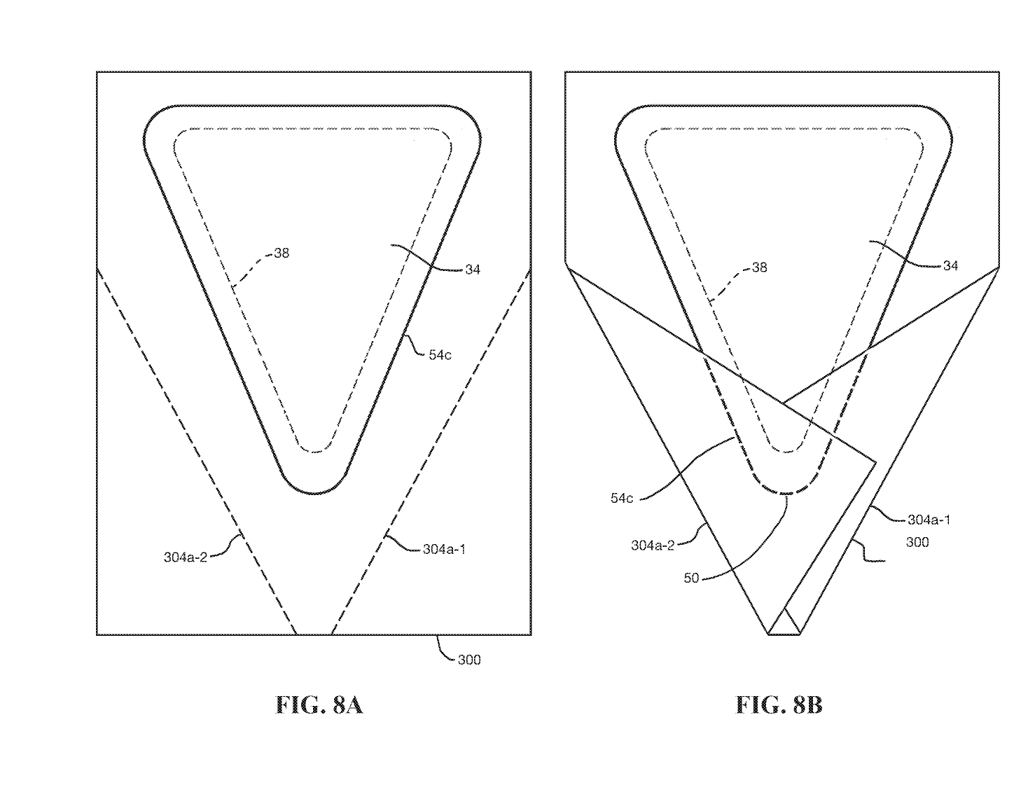

FIGS. 8A and 8B depict an embodiment of the present methods for forming the pad of FIG. 7. In this embodiment, a sheet 300 of material forming backsheet 30 is oversized relative to perimeter 54c of the pad, and sheet 300 is folded along lines 304a-1, 304a-2, as indicated by arrows 308, such that the folded portions of sheet 300 overlap each other and the posterior end of the core as shown in FIG. 8B. Sheet 300 is coupled, for example bonded with adhesive, to topsheet 34 in the region between perimeter 54c and core 38 but left unattached over core 38 to define a “cup” or “pocket” between the sheet 300 and topsheet 34; and sheet 300 is cut, for example die cut, along perimeter 54c to define the pad.

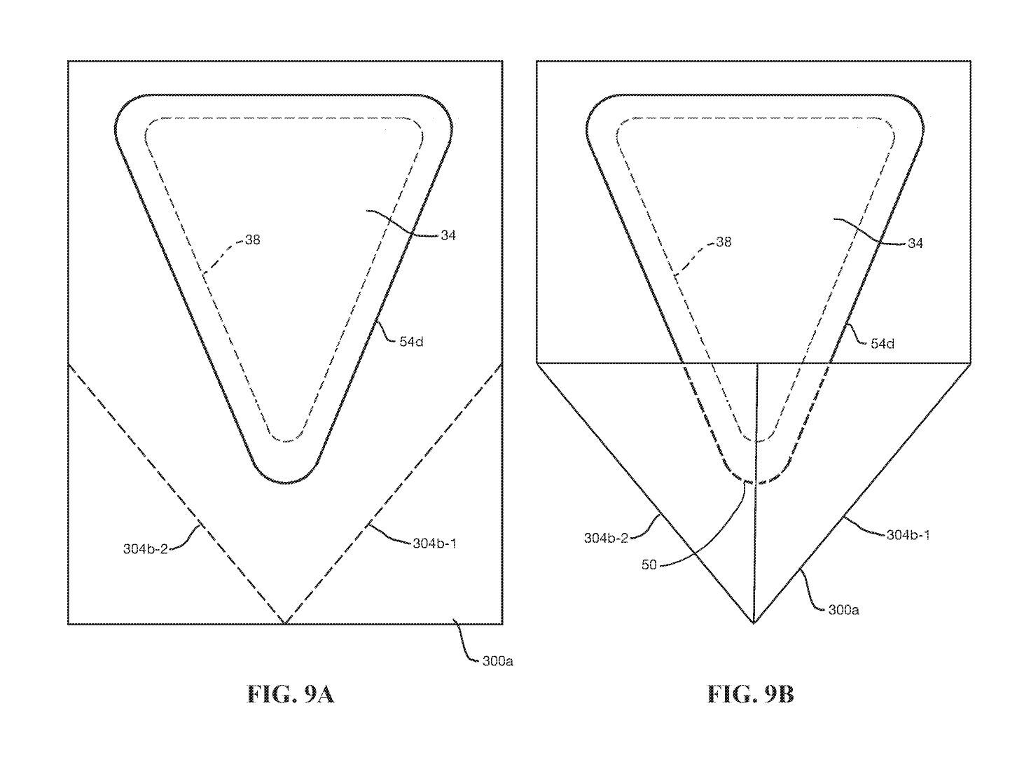

FIGS. 9A and 9B depict a second embodiment of the present methods form forming a first alternate configuration of the present pads with a posterior pocket. In this embodiment, a sheet 300a of material forming backsheet 30 is oversized relative to perimeter 54d of the pad, and sheet 300a is folded along lines 304b-1, 304b-2, as indicated by arrows 308, such that the folded portions of sheet 300a each overlap a portion of the posterior end of the core, and such that the folded portions of sheet 300a abut each other, as shown in FIG. 9B. Sheet is coupled, for example bonded with adhesive, to topsheet 34 in the region between perimeter 54d and core 38 but left unattached over core 38 to define a “cup” or “pocket” between the sheet 300 and topsheet 34; and sheet 300a is cut, for example die cut, along perimeter 54d to define the pad.

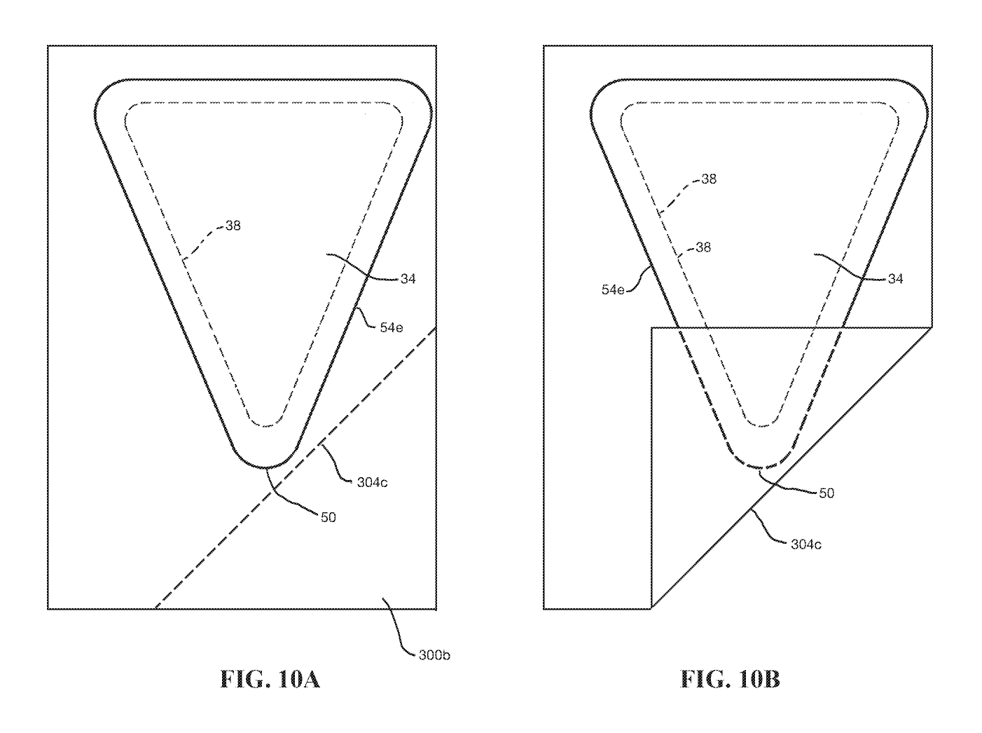

FIGS. 10A and 10B depict a second embodiment of the present methods form forming a second alternate configuration of the present pads with a posterior pocket. In this embodiment, a sheet 300b of material forming backsheet 30 is oversized relative to perimeter 54e of the pad, and sheet 300b is folded along a single line 304c, as indicated by arrow 308, such that the folded portion of sheet 300b overlaps the posterior end of the core, as shown in FIG. 10B. Sheet 300b is coupled, for example bonded with adhesive, to topsheet 34 in the region between perimeter 54e and core 38 but left unattached over core 38 to define a “cup” or “pocket” between the sheet 300 and topsheet 34; and sheet 300b is cut, for example die cut, along perimeter 54e to define the pad.

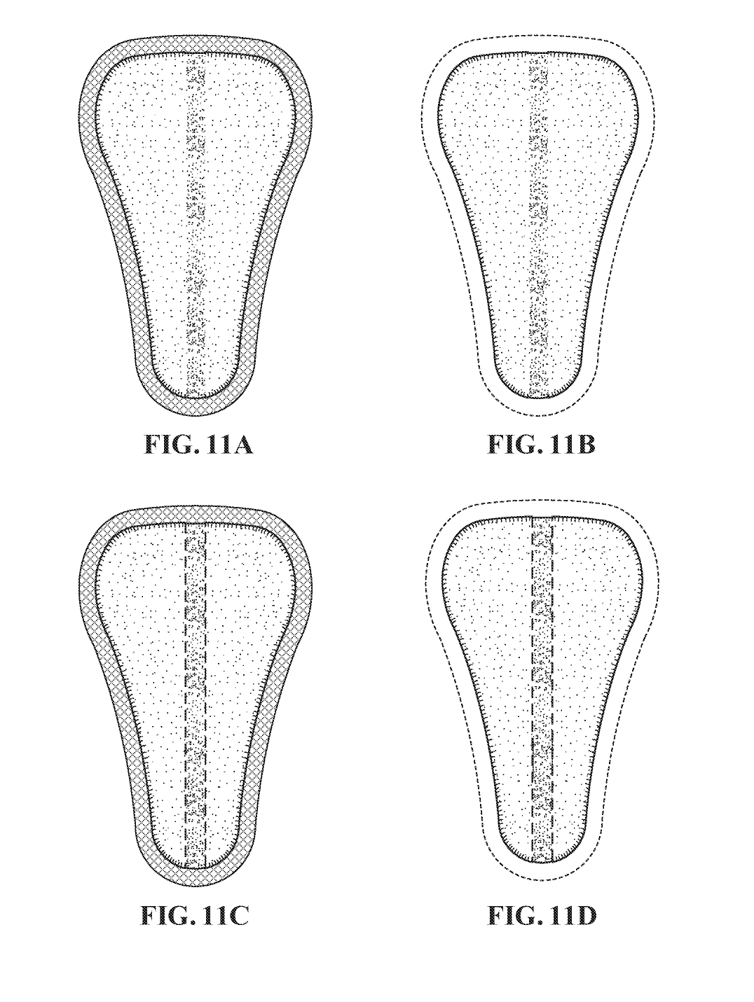

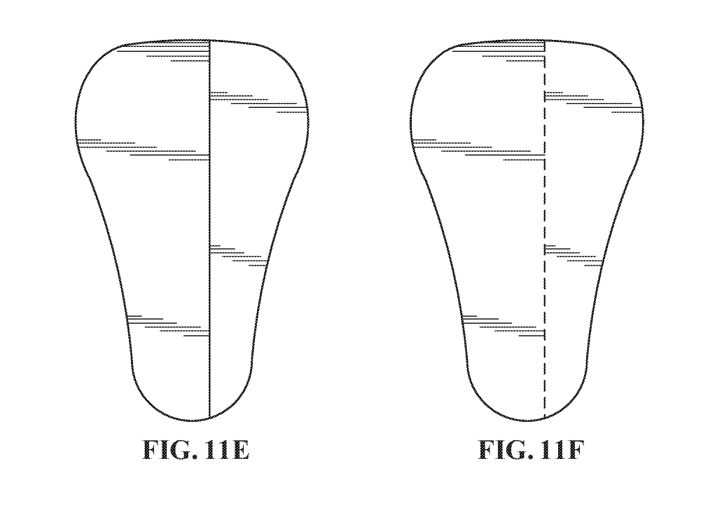

FIGS. 11A-11F depict plan views of designs upper and lower sides of the present pads. In each of FIGS. 11A-11E, dashed lines do not comprise part of the respective design depicted; i.e., dashed lines depict features that are not required for the design. For example, in the design of FIG. 11F, the release liner may have only a single piece. In some embodiments of the present pad designs, any one of the designs of an upper or lower designs FIGS. 11A-11F may stand alone, in which case an individual design does not include opposing side. In other embodiments of the present pad designs, a design of an upper side of any of FIGS. 11A-11D may also include a design of a lower side of any of FIGS. 11E-11F.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A disposable absorbent article comprising:
a pad configured to be inserted into a crotch portion of a wearer's garment or undergarment, the pad comprising:
a liquid-permeable topsheet;
a liquid-impermeable backsheet; and
an absorbent core disposed between the topsheet and the backsheet,
where the pad has a body-facing surface defined by the topsheet, an outer surface defined by the backsheet, an anterior end, a posterior end, a perimeter, a length measured along a central longitudinal axis of the pad between the anterior and posterior ends, and a maximum width measured perpendicular to the length along the body-facing surface, the maximum width disposed closer to the anterior end than to the posterior end;
where the anterior end includes a first lobe on a left side of the central longitudinal axis, and a second lobe on a right side of the central longitudinal axis; and
where the posterior end extends from the anterior end by a distance that is greater than the maximum width.

2. The article of claim 1, where a portion of the perimeter defining the anterior end includes:
a first arc on a left side of the pad, and
a second arc on a right side of the pad,
where the first arc and second arc are separated along the perimeter on the anterior end by at least one additional arc or line.

3. The article of claim 1, where a portion of the perimeter defining the posterior end includes a third arc that spans the central longitudinal axis.

4. The article of any of claims 1-3, where the perimeter includes a left side segment extending between the first arc and the posterior end, and a right side segment extending between the second arc and the posterior end.

5. The article of claim 4, where the left and right side segments are concave toward the central longitudinal axis.

6. The article of any of claims 1-5, where the posterior end defines a third lobe.

7. A disposable absorbent article comprising:
a pad configured to be inserted into a crotch portion of a wearer's garment or undergarment, the pad comprising:
a liquid-permeable topsheet;
a liquid-impermeable backsheet; and
an absorbent core disposed between the topsheet and the backsheet,
where the pad has a body-facing surface defined by the topsheet, an outer surface defined by the backsheet, an anterior end, a posterior end, a perimeter, a length measured along a central longitudinal axis of the pad between the anterior and posterior ends, and a maximum width measured perpendicular to the length, the maximum width disposed closer to the anterior end than to the posterior end; and
where the posterior end extends from the anterior end to define the length; and
where the length is 1 to 2.5 times the maximum width.

8. The article of claim 7, where the length is 1.75 to 2.25 times the maximum width, and the maximum width is closer to the anterior end than to the posterior end.

9. The article of claim 7, where the length is 1 to 1.5 times the maximum width.

10. The article of any of claims 7-9, where the maximum width is disposed at a distance from the anterior end of 1%-25% of the length of the pad.

11. The article of claim 10, where the maximum width is disposed at a distance from the anterior end of 15%-20% of the length of the pad.

12. The article of claim 10, where the maximum width is disposed at a distance from the anterior end of 5%-10% of the length of the pad.

13. The article of any of claims 1-9, where the absorbent core comprises a laminate of superabsorbent polymer (SAP) particles sandwiched between a first layer and a second layer, where at least one of the first and second layers is hydrophilic.

14. The article of claim 13, where the laminate is folded.

15. The article of any of claims 1-14, where the pad further comprises:
an acquisition-distribution layer (ADL) disposed between the topsheet and the absorbent core.

16. A disposable absorbent article comprising:
a pad configured to be inserted into a crotch portion of a wearer's garment or undergarment, the pad comprising:
a liquid-permeable topsheet;
a liquid-impermeable backsheet; and
an absorbent core disposed between the topsheet and the backsheet,
where the pad has a body-facing surface defined by the topsheet, an outer surface defined by the backsheet, an anterior end, a posterior end, a perimeter, a length measured along a central longitudinal axis of the pad between the anterior and posterior ends, and a maximum width measured perpendicular to the length, the maximum width disposed closer to the anterior end than to the posterior end; and
where a first portion of the backsheet on a left side of the pad is folded over the posterior end, and a second portion of the backsheet on a right side of the pad is folded over the posterior end, such that the first and second portions of the backsheet cooperate with the topsheet to define a pocket extending from the posterior end and opening toward the anterior end of the pad.

17. The article of claim 16, where the first portion of the backsheet is bonded to the second portion of the backsheet.

18. The article of claim 16, where the absorbent core comprises a laminate of superabsorbent polymer (SAP) particles sandwiched between a first layer and a second layer, where at least one of the first and second layers is hydrophilic.

19. The article of claim 18, where the laminate is folded.

20. The article of any of claims 16-19, where the pad further comprises:
an acquisition-distribution layer (ADL) disposed between the topsheet and the absorbent core.

21. The article of any of claims 1-20, where the absorbent core has a maximum thickness and defines one or more channels each having a thickness that is less than the maximum thickness.

22. The article of claim 21, where the maximum thickness is defined by a plurality of layers of an absorbent sheet or web.

23. The article of claim 22, where the one or more channels are each defined by embossed lines or regions at which the plurality of layers are compressed.
